(19) United States
(12) Patent Application Publication
(10) Pub. No.: US 2022/0340519 A1
(43) Pub. Date: Oct. 27, 2022

(54) A PROCESS FOR PURIFYING 4,4'-DICHLORODIPHENYL SULFONE

(71) Applicant: BASF SE, Ludwigshafen am Rhein (DE)

(72) Inventors: Jessica Nadine HAMANN, Ludwigshafen am Rhein (DE); Oliver BEY, Ludwigshafen am Rhein (DE); Petra DECKERT, Ludwigshafen am Rhein (DE); Andreas MELZER, Ludwigshafen am Rhein (DE); Christian SCHUETZ, Ludwigshafen am Rhein (DE); Stefan BLEI, Ludwigshafen am Rhein (DE); Frauke THRUN, Ludwigshafen am Rhein (DE)

(21) Appl. No.: 17/635,529

(22) PCT Filed: Aug. 20, 2020
(86) PCT No.: PCT/EP2020/073375

§ 371 Date: Feb. 15, 2022

(30) Foreign Application Priority Data
Aug. 27, 2019 (EP) 19193677.2

(51) Int. Cl. C07C 315/06; C08G 75/23

(52) U.S. Cl. CPC C07C 315/06; C08G 75/23

(57) ABSTRACT

The invention relates to a process for purifying 4,4′-dichlorodiphenyl sulfone comprising: (a) providing a suspension comprising particulate 4,4′-dichlorodiphenyl sulfone in carboxylic acid, (b) carrying out a solid-liquid separation of the suspension to obtain residual moisture containing 4,4′-dichlorodiphenyl sulfone and a carboxylic acid comprising filtrate, (c) washing the residual moisture containing 4,4′-dichlorodiphenyl sulfone with an aqueous base and then with water, (d) mixing the aqueous base after being used for washing with a strong acid, or mixing the aqueous base after being used for washing, the carboxylic acid comprising filtrate and a strong acid, (e) carrying out a phase separation in which an aqueous phase and an organic phase comprising the carboxylic acid are obtained.

The invention relates to a process for purifying 4,4′-dichlorodiphenyl sulfone by solid-liquid separation of a suspension comprising 4,4′-dichlorodiphenyl sulfone in a carboxylic acid and washing the moist 4,4′-dichlorodiphenyl sulfone obtained in the solid-liquid separation.

4,4′-dichlorodiphenyl sulfone (in the following DCDPS) is used for example as a monomer for preparing polymers like polyether sulfone or polysulfone or as an intermediate of pharmaceuticals, dyes and pesticides.

DCDPS for example is produced by oxidation of 4,4′-dichlorodiphenyl sulfoxide which can be obtained by a Friedel-Crafts reaction of thionyl chloride and chlorobenzene as starting materials in the presence of a catalyst, for example aluminum chloride.

CN-A 108047101, CN-A 102351758, CN-B 104402780 and CN-A 104557626 disclose a two-stage process in which in a first stage a Friedel-Crafts acylation reaction is carried out to produce 4,4′-dichlorodiphenyl sulfoxide and in a second stage the 4,4′-dichlorodiphenyl sulfoxide is oxidized to obtain DCDPS in the presence of hydrogen peroxide. The oxidation reaction thereby is carried out in the presence of acetic acid. Such a process in which 4,4′-dichloro-diphenyl sulfoxide is produced in a first stage and DCDPS is obtained in a second stage using hydrogen peroxide in excess and acetic acid as solvent also is described in SU-A 765262.

Further processes for obtaining DCDPS by reacting chlorobenzene and thionyl chloride in a Friedel-Crafts reaction in a first stage to obtain 4,4′-dichlorodiphenyl sulfoxide and to oxidize the 4,4′-dichlorodiphenyl sulfoxide in a second stage using hydrogen peroxide as oxidizing agent and dichloromethane or dichloropropane as solvent are disclosed in CN-A 102351756 and CN-A 102351757.

A process for producing an organic sulfone by oxidation of the respective sulfoxide in the presence of at least one peroxide is disclosed in WO-A 2018/007481. The reaction thereby is carried out in a carboxylic acid as solvent, the carboxylic acid being liquid at 40° C. and having a miscibility gap with water at 40° C. and atmospheric pressure.

In all of these processes the DCDPS containing reaction product is cooled after the reaction is completed to precipitate solid DCDPS and to separate the solid DCDPS from the mixture.

It is an object of the present invention to provide a process for purifying DCDPS by which DCDPS in high purity is achieved and which is environmentally sustainable.

This object is achieved by a process for purifying 4,4′-dichlorodiphenyl sulfone comprising:
(a) providing a suspension comprising particulate 4,4′-dichlorodiphenyl sulfone in carboxylic acid,
(b) carrying out a solid-liquid separation of the suspension to obtain residual moisture containing 4,4′-dichlorodiphenyl sulfone and a carboxylic acid comprising filtrate,
(c) washing the residual moisture containing 4,4′-dichlorodiphenyl sulfone with an aqueous base and then with water,
(d) mixing the aqueous base after being used for washing with a strong acid, or mixing the aqueous base after being used for washing, at least a part of the carboxylic acid comprising filtrate and a strong acid,
(e) carrying out a phase separation in which an aqueous phase and an organic phase comprising the carboxylic acid are obtained.

By washing the residual moisture containing DCDPS (in the following termed as “moist DCDPS”) with an aqueous base and subsequently with water carboxylic acid which is comprised in the moist DCDPS and impurities which may attach to the surface of the crystallized DCDPS can be removed. By washing with an aqueous base, the anions of the carboxylic acid react with the cations of the aqueous base forming an organic salt. A part of this organic salt is removed with the aqueous base during washing with the aqueous base. The rest of the organic salt remains in the moist DCDPS and is removed from the moist DCDPS by the subsequent washing with water.

To reduce the amount of carboxylic acid which is withdrawn from the process and disposed, the aqueous base after being used for washing is mixed with the strong acid or alternatively the aqueous base after being used for washing and at least a part of the carboxylic acid comprising filtrate are mixed with a strong acid. By mixing the aqueous base after being used for washing with the strong acid or by mixing the aqueous base after being used for washing, at least a part of the carboxylic acid comprising filtrate and the strong acid, the anion of the organic salt reacts with the cation of the strong acid and the cation of the organic salt reacts with the anion of the strong acid, whereby carboxylic acid and an inorganic salt are formed. This allows reducing the amount of carboxylic acid which is disposed, because also that part of the carboxylic acid which formed the organic salt during washing with the aqueous base has not to be disposed but can be reused after being separated off. A further advantage of adding the strong acid after washing and thus forming the carboxylic acid and the inorganic salt and reusing of the carboxylic acid is that the total organic carbon (TOC) in the aqueous phase is reduced and thus the aqueous phase is easier to dispose. Preferably, the amounts of aqueous base used for washing and strong acid added to the aqueous base after the aqueous base was used for washing are equimolar.

The suspension comprising particulate DCDPS in a carboxylic acid (in the following termed as “suspension”) for example can derive from the crystallization process in which an organic mixture comprising DCDPS and the carboxylic acid is cooled to a temperature below the saturation point of DCDPS in the organic mixture and the DCDPS starts to crystallize due to cooling.

The saturation point denotes the temperature of the organic mixture at which DCDPS starts to crystallize. This temperature depends on the concentration of the DCDPS in the organic mixture. The lower the concentration of DCDPS in the organic mixture, the lower is the temperature at which crystallization starts.

Besides from a crystallization process, the suspension also can be produced by mixing particulate DCDPS and the carboxylic acid. Such a mixing may be performed for example if particulate DCDPS shall be further purified.

The cooling for crystallizing DCDPS can be carried out in any crystallization apparatus or any other apparatus which allows cooling of the organic mixture, for example an apparatus with surfaces that can be cooled such as a vessel or tank with cooling jacket, cooling coils or cooled baffles like so-called “power baffles”.

Cooling of the organic mixture for crystallization of the DCDPS can be performed either continuously or batchwise. To avoid precipitation and fouling on cooled surfaces, it is preferred to carry out the cooling in a gastight closed vessel by mixing the organic mixture with water in the gastight closed vessel to obtain a liquid mixture and cooling the liquid mixture to a temperature below the saturation point of 4,4′-dichlorodiphenyl sulfone by
(i) reducing the pressure in the gastight closed vessel to a pressure at which the water starts to evaporate,
(ii) condensing the evaporated water by cooling
(iii) mixing the condensed water into the liquid mixture in the gastight closed vessel to obtain a suspension comprising crystallized 4,4′-dichlorodiphenyl sulfone;

This process allows for cooling the DCDPS comprising organic mixture without cooling surfaces onto which particularly at starting the cooling process crystallized DCDPS accumulates and forms a solid layer. This enhances the efficiency of the cooling process. Also, additional efforts to remove this solid layer can be avoided.

If cooling is performed according to this process, the suspension which is subjected to the solid-liquid separation additionally contains water besides the crystallized DCDPS and the carboxylic acid.

Particularly when carboxylic acids are used as solvent which have a boiling point above 150° C. at 1 bar, cooling by reducing the pressure to evaporate solvent, to condense the evaporated solvent by cooling and recycling the condensed solvent back into the gastight vessel would require a high energy consumption to achieve the necessary low pressures. Using higher temperatures to evaporate solvent for shifting the saturation point such that DCDPS crystallizes on the other hand would have a negative effect on the DCDPS; particularly a change in color of the DCDPS cannot be excluded. By mixing the organic mixture with water and to evaporate, condense and recycle the condensed water, it is possible to shift the saturation point by cooling without evaporating solvent at high temperatures or to reduce the pressure to very low values which is very energy consuming. Surprisingly, cooling and crystallization of DCDPS by adding water, reducing the pressure to evaporate water, condensing the water by cooling and recycle the condensed water and mix it into the organic mixture even can be carried out when carboxylic acids are used as solvent which have a poor solubility in water.

To crystallize the DCDPS, it is preferred to provide crystal nuclei. To provide the crystal nuclei, it is possible to use dried crystals which are added to the organic mixture or to add a suspension comprising particulate DCDPS as crystal nuclei. If dried crystals are used but the crystals are too big, it is possible to grind the crystals into smaller particles which can be used as crystal nuclei. Further, it is also possible to provide the necessary crystal nuclei by applying ultrasound to the liquid mixture. Preferably, the crystal nuclei are generated in situ in an initializing step. The initializing step preferably comprises following steps before reducing pressure in step (i):
reducing the pressure in the gastight closed vessel such that the boiling point of the water in the liquid mixture is in the range from 80 to 95° C.;
evaporating water until an initial formation of solids takes place;
increasing the pressure in the vessel and heating the liquid mixture in the gastight closed vessel to a temperature in the range from 1 to 10° C. below the saturation point of DCDPS.

By reducing the pressure in the vessel such that the water starts to evaporate at a temperature in the range from 80 to 95° C., more preferred in the range from 83 to 92° C., the following evaporation of water leads to a saturated solution and the precipitation of DCDPS. By the following pressure increase and heating the organic mixture in the gastight closed vessel to a temperature in the range from 1 to 10° C. below the saturation point of DCDPS the solidified DCDPS starts to partially dissolve again. This has the effect that the number of crystal nuclei is reduced which allows producing a smaller amount of crystals with a bigger size. Further it is ensured that an initial amount of crystal nuclei remains in the gastight closed vessel. Cooling, particularly by reducing the pressure, can be started immediately after a pre-set temperature within the above ranges is reached to avoid complete dissolving of the produced crystal nuclei. However, it is also possible to start cooling after a dwell time for example of 0.5 to 1.5 h at the pre-set temperature.

For generating the crystal nuclei in the initializing step, it is possible to only evaporate water until an initial formation of solids take place. It is also possible to entirely condense the evaporated water by cooling and to return all the condensed water into the gastight closed vessel.

The latter has the effect that the organic mixture in the gastight closed vessel is cooled and solid forms. A mixture of both approaches, where only a part of the evaporated and condensed water is returned into the gastight vessel, is also viable.

Cooling of the organic mixture by reducing the pressure, evaporate water, condense the evaporated water by cooling and mixing the condensed water into the liquid mixture can be carried out batchwise, semi-continuously or continuously.

Particularly in a batchwise process, the pressure reduction to evaporate water and thereby to cool the organic mixture can be for example stepwise or continuously. If the pressure reduction is stepwise, it is preferred to hold the pressure in one step until a predefined rate in temperature decrease can be observed, particularly until the predefined rate is “O” which means that no further temperature decrease occurs. After this state is achieved, the pressure is reduced to the next pressure value. In this case the steps for reducing the pressure all can be the same or can be different. If the pressure is reduced in different steps, it is preferred to reduce the size of the steps with decreasing pressure. Preferably, the steps in which the pressure is decreased are in a range from 10 to 800 mbar, more preferred in a range from 30 to 500 mbar and particularly in a range from 30 to 300 mbar.

If the pressure reduction is continuously, the pressure reduction can be for example linearly, hyperbolic, parabolic or in any other shape, wherein it is preferred for a non-linear decrease in pressure to reduce the pressure in such a way that the pressure reduction decreases with decreasing pressure. If the pressure is reduced continuously, it is preferred to reduce the pressure with a rate from 130 to 250 mbar/h, particularly with a rate from 180 to 220 mbar/h. Moreover, the pressure can be reduced bulk temperature controlled by use of a process control system (PCS), whereby a stepwise linear cooling profile is realized.

Preferably, the pressure reduction is temperature controlled with a stepwise cooling profile from 5 to 25 K/h to approximate a constant supersaturation with increasing solid content and thus, more crystalline surface for growth.

If the cooling and thereby the crystallization is carried out in a semi-continuous process, the pressure preferably is reduced stepwise, wherein the semi-continuous process for example can be realized by using at least one gastight vessel for each pressure step, respectively temperature step. For cooling the organic mixture, the organic mixture is fed into the first gastight vessel having the highest temperature and cooled to a first temperature. Then the organic mixture is withdrawn from the first gastight vessel and fed into a second gastight vessel having a lower pressure. This process is repeated until the liquid mixture is fed into the gastight vessel having the lowest pressure. As soon as the liquid mixture is withdrawn from one vessel, fresh organic mixture can be fed into that vessel, wherein the pressure in the vessel preferably is kept constant. “Constant” in this context means that variations in pressure which depend on withdrawing and feeding liquid mixture into the respective tank are kept as low as technically possible but cannot be excluded.

Besides carrying out the process batchwise or semi-continuous, it is also possible to perform the process continuously. If the cooling and thus the crystallization of DCDPS is performed continuously, it is preferred to operate the cooling and crystallization stepwise in at least two steps, particularly in two to three steps, wherein for each step at least on gastight closed vessel is used. If the cooling and crystallization is carried out in two steps, in a first step the organic mixture preferably is cooled to a temperature in the range from 40 to 90° C. and in a second step preferably to a temperature in the range from −10 to 50° C. If the cooling is operated in more than two steps, the first step preferably is operated at a temperature in the range from 40 to 90° C. and the last step at a temperature in the range from −10 to 30° C. The additional steps are operated at temperatures between these ranges with decreasing temperature from step to step. If the cooling and crystallization is performed in three steps, the second step for example is operated at a temperature in the range from 10 to 50° C.

If the cooling and crystallization is carried out continuously, a stream of the suspension is continuously withdrawn from the last gastight vessel. The suspension then is fed into the solid-liquid-separation (b). To keep the liquid level in the gastight closed vessels within predefined limits fresh organic mixture comprising DCDPS, carboxylic acid and water can be fed into each gastight closed vessel in an amount corresponding or essentially corresponding to the amount of suspension withdrawn from the respective gastight closed vessel. The fresh organic mixture either can be added continuously or batchwise each time a minimum liquid level in the gastight closed vessel is reached.

Independently of being carried out batchwise or continuously, crystallization preferably is continued until the solids content in the suspension in the last step of the crystallization is in the range from 5 to 50 wt %, more preferred in the range from 5 to 40 wt % and particularly in the range from 20 to 40 wt %, based on the mass of the suspension.

To achieve this solids content in the suspension, it is preferred to reduce the pressure in (i) until the suspension which is obtained by the cooling has cooled down to a temperature in the range from 10 to 30° C., preferably in the range from 15 to 30° C. and particularly in the range from 20 to 30° C.

The pressure at which this temperature is achieved depends on the amount of water in the organic mixture. Preferably, the amount of water mixed to the organic mixture is such that the amount of water in the organic mixture is in the range from 10 to 60 wt % based on the total amount of the organic mixture. More preferred, the amount of water mixed to the organic mixture is such that the amount of water in the organic mixture is in the range from 10 to 50 wt % based on the total amount of the organic mixture and, particularly, the amount of water mixed to the organic mixture is such that the amount of water in the organic mixture is in the range from 15 to 35 wt % based on the total amount of the organic mixture.

Even though the cooling and crystallization can be carried out continuously or batchwise, it is preferred to carry out the cooling and crystallization batchwise. Batchwise cooling and crystallization allows a higher flexibility in terms of operating window and crystallization conditions and is more robust against variations in process conditions.

To support cooling of the organic mixture it is further possible to provide the gastight closed vessel with coolable surfaces for an additional cooling. The coolable surfaces for example can be a cooling jacket, cooling coils or cooled baffles like so called “power baffles”. Surprisingly, forming of precipitations and fouling on coolable surfaces can be avoided or at least considerably reduced, if the additional cooling is started not before the temperature of the organic mixture is reduced to a temperature in the range from 20 to 60° C., more preferred in a range from 20 to 50° C. and particularly in a range from 20 to 40° C.

The organic mixture comprising the DCDPS and carboxylic acid can be obtained by any process known to a skilled person. This organic mixture for example can be produced by mixing DCDPS and carboxylic acid, for example for purifying DCDPS by the inventive process. Preferably, the organic mixture is obtained by an oxidization reaction of 4,4′-dichlorodiphenyl sulfoxide and an oxidization agent which is carried out in the carboxylic acid as solvent.

If the organic mixture is obtained by an oxidization reaction, it is particularly preferred that the DCDPS is produced by reacting a solution comprising 4,4′-dichlorodiphenyl sulfoxide and at least one C₆-C₁₀ carboxylic acid as organic solvent with an oxidizing agent to obtain a crude reaction product comprising 4,4′-dichlorodiphenyl sulfone, wherein the concentration of water in the reaction mixture is kept below 5 wt %.

By keeping the concentration of water below 5 wt % it is possible to use the linear C₆-C₁₀ carboxylic acid which is only slightly health hazardous and which has a good biodegradability.

Another advantage of using the linear C₆-C₁₀ carboxylic acid is that the linear C₆-C₁₀ carboxylic acid shows a good separability from water at low temperatures which allows separation of the linear C₆-C₁₀ carboxylic acid without damaging the product and which further allows recycling the linear C₆-C₁₀ carboxylic acid as solvent into the oxidation process.

In the process for producing DCDPS a solution comprising 4,4′-dichlorodiphenyl sulfoxide (in the following termed as DCDPSO) and at least one C₆-C₁₀ carboxylic acid (in the following termed as carboxylic acid) is provided. In this solution, the carboxylic acid serves as solvent. Preferably, the ratio of DCDPSO to carboxylic acid is in a range from 1:2 to 1:6, particularly in a range from 1:2.5 to 1:3.5. Such a ratio of DCDPSO to carboxylic acid is usually sufficient to completely solve the DCDPSO in the carboxylic acid at the reaction temperature and to achieve an almost full conversion of the DCDPSO forming DCDPS and further to use as little carboxylic acid as possible. The solution comprising DCDPSO and carboxylic acid preferably is heated to a temperature in the range from 70 to 110° C., more preferred to a temperature in the range from 80 to 100° C. and particularly in the range from 85 to 95° C., for example 86, 87, 88, 89, 90, 91, 92, 93, 94° C., before adding the oxidizing agent.

To provide the solution, it is possible to feed DCDPSO and the carboxylic acid separately into a reactor and to mix the DCDPSO and the carboxylic acid in the reactor. Alternatively, it is also possible to mix the DCDPSO and the carboxylic acid in a separate mixing unit to obtain the solution and to feed the solution into the reactor. In a further alternative, DCDPSO and a part of the carboxylic acid are fed into the reactor as a mixture and the rest of the carboxylic acid is fed directly into the reactor and the solution is obtained by mixing the mixture of DCDPSO and part of the carboxylic acid and the rest of the carboxylic acid in the reactor.

The at least one carboxylic acid used in the reaction preferably is the same as used as solvent in the organic mixture and the suspension provided in (a) and can be only one carboxylic acid or a mixture of at least two different carboxylic acids. Preferably the carboxylic acid is at least one aliphatic carboxylic acid. The at least one aliphatic carboxylic acid may be at least one linear or at least one branched aliphatic carboxylic acid or it may be a mixture of one or more linear and one or more branched aliphatic carboxylic acids. Preferably the aliphatic carboxylic acid is a C₆ to C₉ carboxylic acid, whereby it is particularly preferred that the at least one carboxylic acid is an aliphatic monocarboxylic acid. Thus, the at least one carboxylic acid may be hexanoic acid, heptanoic acid, octanoic acid nonanoic acid or decanoic acid or a mixture of one or more of said acids. For instance, the at least one carboxylic acid may be n-hexanoic acid, 2-methyl-pentanoic acid, 3-methyl-pentanoic acid, 4-methyl-pentanoic acid, n-heptanoic acid, 2-methyl-hexanoic acid, 3-methyl-hexanoic acid, 4-methyl-hexanoic acid, 5-methyl-hexanoic acid, 2-ethyl-pentanoic acid, 3-ethyl-pentanoic acid, n-octanoic acid, 2-methyl-heptanoic acid, 3-methyl-heptanoic acid, 4-methyl-heptanoic acid, 5-methyl-heptanoic acid, 6-methyl-heptanoic acid, 2-ethyl-hexanoic acid, 4-ethyl-hexanoic acid, 2-propyl pentanoic acid, 2,5-dimethylhexanoic acid, 5,5-dimethyl-hexanoic acid, n-nonanoic acid, 2-ethyl-heptanoic acid, n-decanoic acid, 2-ethyl-octanoic acid, 3-ethyl-ocantoic acid, 4-ethyl-octanoic acid. The carboxylic acid may also be a mixture of different structural isomers of one of said acids. For instance, the at least one carboxylic acid may be isononanoic acid comprising a mixture of 3,3,5-trimethyl-hexanoic acid, 2,5,5-trimethyl-hexanoic acid and 7-methyl-octanoic acid or neodecanoic acid comprising a mixture of 7,7-dimethyloctanoic acid, 2,2,3,5-tetramethyl-hexanoic acid, 2,4-dimethyl-2-isopropylpentanoic acid and 2,5-dimethyl-2-ethylhexanoic acid. Particularly preferably, however, the carboxylic acid is a linear C₆-C₁₀ carboxylic acid and particularly n-hexanoic acid or n-heptanoic acid.

Heating of the solution comprising DCDPSO and the carboxylic acid can be carried out in the reactor in which the reaction for obtaining the crude reaction product takes place or in any other apparatus before being fed into the reactor. Particularly preferably, the solution comprising DCDPSO and the carboxylic acid is heated to the respective temperature before being fed into the reactor. Heating of the solution for example can be carried out in a heat exchanger through which the solution flows before being fed into the reactor or more preferred in a buffer container in which the solution is stored before being fed into the reactor. If such a buffer container is used, the buffer container also may serve as mixing unit for mixing the DCDPSO and the carboxylic acid to obtain the solution.

A heat exchanger for example can be used when the process is operated continuously. Heating of the solution in a buffer container can be carried out in a continuously operated process as well as in a batchwise operated process. If a heat exchanger is used for heating the solution, any suitable heat exchanger can be used, for example a shell and tube heat exchanger, a plate heat exchanger, a spiral tube heat exchanger, or any other heat exchanger known to a skilled person. The heat exchanger thereby can be operated in counter current flow, co-current flow or cross flow.

Besides heating by using a heating fluid which usually is used in a heat exchanger or for heating in a double jacket or heating coil, also electrical heating or induction heating can be used for heating the solution.

If the solution is heated in the buffer container, any suitable container which allows heating of the contents in the container can be used. Suitable containers for example are equipped with a double jacket or a heating coil. If the buffer container additionally is used for mixing the DCDPSO and the carboxylic acid, the buffer container further comprises a mixing unit, for example a stirrer.

For carrying out the reaction, the solution preferably is provided in a reactor. This reactor can be any reactor which allows mixing and reacting of the components fed into the reactor. A suitable reactor for example is a stirred tank reactor or a reactor with forced circulation, particularly a reactor with external circulation and a nozzle to feed the circulating liquid. If a stirred tank reactor is used, any stirrer can be used. Suitable stirrers for example are axially conveying stirrers like oblique blade agitators or cross-arm stirrers or radially conveying agitators like flat blade agitators. The stirrer may have at least 2 blades, more preferred at least 4 blades. Particularly preferred is a stirrer having 4 to 8 blades, for example 6 blades. For reasons of process stability and process reliability, it is preferred that the reactor is a stirred tank reactor with an axially conveying stirrer.

For controlling the temperature in the reactor, it is further preferred to use a reactor with heat exchange equipment, for example a double jacket or a heating coil. This allows additional heating or heat dissipation during the reaction and keep the temperature constant or in a predefined temperature range at which the reaction is carried out. Preferably, the reaction temperature is kept in a range from 70 to 110° C., more preferred from 80 to 100° C. and particularly in a range from 85 to 95° C., for example 86, 87, 88, 89 90, 91, 92, 93, 94° C.

To obtain DCDPS, the solution comprising DCDPSO and carboxylic acid is oxidized by an oxidizing agent. Therefore, the oxidizing agent preferably is added to the solution to obtain a reaction mixture. From the reaction mixture the crude reaction product comprising DCDPS can be obtained.

The oxidizing agent used for oxidizing DCDPSO for obtaining DCDPS preferably is at least one peroxide. The at least one peroxide may be at least one peracid, for example one or a mixture of two or more, such as three or more peracids. Preferably, the process disclosed herein is carried out in the presence of one or two, particularly in the presence of one peracid. The at least one peracid may be a C₁ to C₁₀ peracid, which may be unsubstituted or substituted, e.g. by linear or branched C₁ to C₅ alkyl or halogen, such as fluorine. Examples thereof are peracetic acid, performic acid, perpropionic acid, percaprionic acid, pervaleric acid or pertrifluoroacetic acid.

Particularly preferably the at least one peracid is a C₆ to C₁₀ peracid, for example 2-ethyl-hexanoic peracid. If the at least one peracid is soluble in water, it is advantageous to add the at least one peracid as aqueous solution. Further, if the at least one peracid is not sufficiently soluble in water, it is advantageous that the at least one peracid is dissolved in the respective carboxylic acid. Most preferably, the at least one peracid is a linear C₆ to C₁₀ peracid which is generated in situ.

Particularly preferably, the peracid is generated in situ by using hydrogen peroxide (H₂O₂) as oxidizing agent. At least a part of the added H₂O₂ reacts with the carboxylic acid forming the peracid. The H₂O₂ preferably is added as an aqueous solution, for instance of 1 to 90 wt % solution, such as a 20, 30, 40, 50, 60 or 70 wt % solution, preferably as 30 to 85 wt % solution, particularly as a 50 to 85 wt % solution, each being based on the total amount of the aqueous solution. Using a highly concentrated aqueous solution of H₂O₂, particularly a solution of 50 to 85 wt %, for example of 70 wt %, based on the total amount of the aqueous solution, may lead to a reduction of reaction time. It may also facilitate recycling of the at least one carboxylic acid.

Particularly preferably, the at least one peracid is a linear C₆ or C₇ peracid which is generated in situ. To additionally reduce the reaction time and to add only a small amount of water to the reaction mixture, it is particularly preferred that the C₆-C₁₀ carboxylic acid is n-hexanoic acid or n-heptanoic acid and the hydrogen peroxide is a 50 to 85 wt % solution.

To avoid accumulation of the oxidizing agent and to achieve a constant oxidation of the DCDPSO, it is preferred to add the oxidizing agent continuously with a feed rate from 0.002 to 0.01 mol per mol DCDPSO and minute. More preferred, the oxidizing agent is added with a feed rate from 0.003 to 0.008 mol per mol DCDPSO and minute and particularly with a feed rate from 0.004 to 0.007 mol per mol DCDPSO and minute.

The oxidizing agent can be added with a constant feed rate or with a varying feed rate. If the oxidizing agent is added with a varying feed rate, it is for example possible to reduce the feed rate with proceeding reaction within the above described range. Further it is possible to add the oxidizing agent in several steps with a stop of adding oxidizing agent between the steps. In each step during adding the oxidizing agent, the oxidizing agent can be added with a constant feed rate or a varying feed rate. Besides a decreasing feed rate with proceeding reaction, it is also possible to increase the feed rate or to switch between increasing and decreasing feed rates. If the feed rate is increased or decreased, the change in feed rate can be continuously or stepwise. Particularly preferably, the oxidizing agent is added in at least two steps wherein the feed rate in each step is constant.

If the oxidizing agent is fed in at least two steps, it is preferred to add the oxidizing agent in two steps, wherein adding the oxidizing agent to the solution preferably comprises:
(A) adding 0.9 to 1.05 mol oxidizing agent per mol 4,4′-dichlorodiphenyl sulfoxide uniformly distributed to the solution at a temperature in the range from 70 to 110° C. over a period from 1.5 to 5 h in a first step to obtain a reaction mixture;
(B) agitating the reaction mixture after completion of the first step at the temperature of the first step for 5 to 30 min without adding oxidizing agent;
(C) adding 0.05 to 0.2 mol oxidizing agent per mol 4,4′-dichlorodiphenyl sulfoxide to the reaction mixture at a temperature in the range from 80 to 110° C. over a period of less than 40 min in a second step;
(D) agitating the reaction mixture after completion of the second step at the temperature of the second step for 10 to 30 min without adding oxidizing agent,
(E) heating the reaction mixture to a temperature in the range from 95 to 110° C. and hold this temperature for 10 to 90 min to obtain a crude reaction product comprising 4,4′-dichlorodiphenyl sulfone.

If the oxidation of DCDPSO is carried out in at least two steps, for converting the DCDPSO into DCDPS, the DCDPSO is oxidized by adding the oxidizing agent in the first and second steps to the solution comprising DCDPSO and carboxylic acid.

In the first step 0.9 to 1.05 mol oxidizing agent per mol 4,4′-dichlorodiphenyl sulfoxide are added uniformly distributed to the solution at a temperature in the range from 70 to 110° C. over a period from 1.5 to 5 h. By adding the oxidizing agent over such a period an accumulation of the oxidizing agent can be avoided.

“Uniformly distributed” in this context means, that the oxidizing agent can be added either continuously at a constant feed rate or at periodically changing feed rates. Besides continuous periodically changing feed rates, periodically changing feed rates also comprise discontinuously changing periodical feed rates for example feed rates where oxidizing agent is added for a defined time, then no oxidizing agent is added for a defined time and this adding and not adding is repeated until the complete amount of oxidizing agent for the first step is added. The period in which the oxidizing agent is added, is in a range from 1.5 to 5 h, more preferred in a range from 2 to 4 h and particularly in a range from 2.5 to 3.5 h. By adding the oxidizing agent uniformly distributed over such a period, it can be avoided that oxidizing agent accumulates in the reaction mixture which may result in an explosive mixture. Additionally, by adding the oxidizing agent over such a period, the process can be scaled up in an easy way as this allows also in an upscaled process to dissipate the heat from the process. On the other hand, by such an amount decomposition of the hydrogen peroxide is avoided and thus the amount of hydrogen peroxide used in the process can be minimized.

The temperature at which the first step is carried out is in the range from 70 to 110° C., preferably in the range from 85 to 100° C. and particularly in the range from 90 to 95° C. In this temperature range, a high reaction velocity can be achieved at high solubility of the DCDPSO in the carboxylic acid. This allows to minimize the amount of carboxylic acid and by this a controlled reaction can be achieved.

After the addition of the oxidizing agent in the first step is completed, the reaction mixture is agitated at the temperature of the first step for 5 to 30 min without adding oxidizing agent. By agitating the reaction mixture after completion of adding the oxidizing agent, oxidizing agent and DCDPSO which did not yet react are brought into contact to continue the reaction forming DCDPS for reducing the amount of DCDPSO remaining as impurity in the reaction mixture.

To further reduce the amount of DCDPSO in the reaction mixture, after completing of agitating without adding oxidizing agent, 0.05 to 0.2 mol oxidizing agent per DCDPSO, preferably 0.06 to 0.15 mol oxidizing agent per mol DCDPSO, and particularly 0.08 to 0.1 mol oxidizing agent per mol DCDPSO are added to the reaction mixture in the second step.

In the second step, the oxidizing agent preferably is added in a period from 1 to 40 min, more preferred in a period from 5 to 25 min and particularly in a period from 8 to 15 min. The addition of the oxidizing agent in the second step may take place in the same way as in the first step. Further, it is also possible to add the entire oxidizing agent of the second step at once.

The temperature of the second step is in the range from 80 to 110° C., more preferred in the range from 85 to 100° C. and particularly in the range from 93 to 98° C. It further is preferred that the temperature in the second step is from 3 to 10° C. higher than the temperature in the first step. More preferred the temperature in the second step is 4 to 8° C. higher than the temperature in the first step and particularly preferably, the temperature in the second step is 5 to 7° C. higher than the temperature in the first step. By the higher temperature in the second step, it is possible to achieve a higher reaction velocity.

After addition of the oxidizing agent in the second step, the reaction mixture is agitated at the temperature of the second step for 10 to 20 min to continue the oxidation reaction of DCDPSO forming DCDPS.

To complete the oxidation reaction, after agitating at the temperature of the second step without adding oxidizing agent, the reaction mixture is heated to a temperature in the range from 95 to 110° C., more preferred in the range from 95 to 105° C. and particularly in the range from 98 to 103° C. and held at this temperature for 10 to 90 min, more preferred from 10 to 60 min and particularly from 10 to 30 min.

In the oxidizing process, particularly when using H₂O₂ as oxidizing agent, water is formed. Further, water may be added with the oxidizing agent. According to the invention, the concentration of the water in the reaction mixture is kept below 5 wt %, more preferred below 3 wt % and particularly below 2 wt %. By using aqueous hydrogen peroxide with a concentration of 70 to 85 wt % the concentration of water during the oxidization reaction is kept low. It even may be possible to keep the concentration of water in the reaction mixture during the oxidization reaction below 5 wt % without removing water by using aqueous hydrogen peroxide with a concentration of 70 to 85 wt %.

Additionally or alternatively, it may be necessary to remove water from the process for keeping the concentration of water in the reaction mixture below 5 wt %. To remove the water from the process, it is for example possible to strip water from the reaction mixture. Stripping thereby preferably is carried out by using an inert gas as stripping medium. If the concentration of water in the reaction mixture remains below 5 wt % when using aqueous hydrogen peroxide with a concentration of 70 to 85 wt % it is not necessary to additionally strip water. However, even in this case it is possible to strip water to further reduce the concentration.

Suitable inert gases which can be used for stripping the water are non-oxidizing gases and are preferably nitrogen, carbon dioxide, noble gases like argon or any mixture of these gases. Particularly preferably, the inert gas is nitrogen.

The amount of inert gas used for stripping the water preferably is in the range from 0 to 2 Nm³/h/kg, more preferably in the range from 0.2 to 1.5 Nm³/h/kg and particularly in the range from 0.3 to 1 Nm³/h/kg. The gas rate in Nm³/h/kg can be determined according to DIN 1343, January 1990 as relative gas flow. Stripping of water with the inert gas may take place during the whole process or during at least one part of the process. If water is stripped at more than one part of the process, between the parts stripping of water is interrupted. The interruption of stripping water is independent of the mode in which the oxidizing agent is added. For example, it is possible to add the oxidizing agent without any interruption and to strip the water with interruptions or to add the oxidizing agent in at least two steps and to strip the water continuously. Further it is also possible, to strip water only during the addition of oxidizing agent. Particularly preferably, the water is stripped by continuously bubbling an inert gas into the reaction mixture.

To avoid the formation of areas with different compositions in the reactor which may lead to different conversion rates of DCDPSO and thus to different yield and amounts of impurities, it is preferred to homogenize the reaction mixture during the first step and the second step. Homogenization of the reaction mixture can be performed by any method known to a skilled person, for example by agitating the reaction mixture. To agitate the reaction mixture, it is preferred to stir the reaction mixture. For stirring, any suitable stirrer can be used. Suitable stirrers for example are axially conveying stirrers like oblique blade agitators or cross-arm stirrers or radially conveying agitators like flat blade agitators. The stirrer may have at least 2 blades, more preferred at least 4 blades. Particularly preferred is a stirrer having 4 to 8 blades, for example 6 blades. For reasons of process stability and process reliability, it is preferred that the reactor is a stirred tank reactor with an axially conveying stirrer.

The temperature of the reaction mixture during the process can be set for example by providing a pipe inside the reactor through which a tempering medium can flow. Under the aspect of ease of reactor maintenance and/or uniformity of heating, preferably, the reactor comprises a double jacket through which the tempering medium can flow. Besides the pipe inside the reactor or the double jacket the tempering of the reactor can be performed in each manner known to a skilled person, for example by withdrawing a stream of the reaction mixture from the reactor, passing the stream through a heat exchanger in which the stream is tempered and recycle the tempered stream back into the reactor.

To support the oxidation reaction, it is further advantageous to additionally add at least one acidic catalyst to the reaction mixture. The acidic catalyst may be at least one, such as one or more, such as a mixture of two or three additional acids. An additional acid in this context is an acid which is not the carboxylic acid which serves as solvent. The additional acid may be an inorganic or organic acid, with the additional acid preferably being an at least one strong acid. Preferably, the strong acid has a pKₐ value from −9 to 3, for instance −7 to 3 in water. A person skilled in the art appreciates that such acid dissociation constant values, Kₐ, can be for instance found in a compilation such as in IUPAC, Compendium of Chemical Terminology, 2^{nd} ed. “Gold Book”, Version 2.3.3, 2014-02-24, page 23. The person skilled in the art appreciates that such pKₐ values relate to the negative logarithm value of the Kₐ value. it is more preferred that the at least one strong acid has a negative pKₐ value, such as from −9 to −1 or −7 to −1 in water.

Examples for inorganic acids being the at least one strong acid are nitric acid, hydrochloric acid, hydrobromic acid, perchloric acid, and/or sulfuric acid. Particularly preferably, one strong inorganic acid is used, in particular sulfuric acid. While it may be possible to use the at least one strong inorganic acid as aqueous solution, it is preferred that the at least one inorganic acid is used neat. Suitable strong organic acids for example are organic sulfonic acids, whereby it is possible that at least one aliphatic or at least one aromatic sulfonic acid or a mixture thereof is used. Examples for the at least one strong organic acid are para-toluene sulfonic acid, methane sulfonic acid or trifluormethane sulfonic acid. Particularly preferably the strong organic acid is methane sulfonic acid. Besides using either at least one inorganic strong acid or at least one organic strong acid, it is also possible to use a mixture of at least one inorganic strong acid and at least one organic strong acid as acidic catalyst. Such a mixture for example may comprise sulfuric acid and methane sulfonic acid.

The acidic catalyst preferably is added in catalytic amounts. Thus, the amount of acidic catalyst used may be in the range from 0.1 to 0.3 mol per mol DCDPSO, more preferred in the range from 0.15 to 0.25 mol per mol DCDPSO. However, it is preferred to employ the acidic catalyst in an amount of less than 0.1 mol per mol DCDPSO, such as in an amount from 0.001 to 0.08 mol per mol DCDPSO, for example from 0.001 to 0.03 mol per mol DCDPSO. Particularly preferably, the acidic catalyst is used in an amount from 0.005 to 0.01 mol per mol DCDPSO.

The oxidization reaction for obtaining DCDPS can be carried out as a batch process, as a semi continuous process or as a continuous process. Preferably, the oxidization reaction is carried out batchwise. The oxidation reaction can be carried out at atmospheric pressure or at a pressure which is below or above atmospheric pressure, for example in a range from 10 to 900 mbar(abs). Preferably, the oxidation reaction is carried out at a pressure in a range from 200 to 800 mbar(abs) and particularly in a range from 400 to 700 mbar(abs).

The oxidization reaction can be carried out under ambient atmosphere or inert atmosphere. If the oxidization reaction is carried out under inert atmosphere, it is preferred to purge the reactor with an inert gas before feeding the DCDPSO and the carboxylic acid. If the oxidization reaction is carried out under an inert atmosphere and the water formed during the oxidation reaction is stripped with an inert gas, it is further preferred that the inert gas used for providing the inert atmosphere and the inert gas which is used for stripping the water is the same. It is a further advantage of using an inert atmosphere that the partial pressure of the components in the oxidization reaction, particularly the partial pressure of water is reduced.

By the oxidization reaction, the organic mixture is obtained which comprises 4,4′-dichlorodiphenyl sulfoxide solved in the at least one carboxylic acid. Therefore, the carboxylic acid of the suspension which is separated off in the solid-liquid separation is the same as used for the production of the DCDPS in the above process.

After completing the cooling and crystallization by pressure reduction, the process is finished and preferably the pressure is set to ambient pressure, again. After reaching ambient pressure, the suspension which formed by cooling the liquid mixture in the gastight vessel is subjected to the solid-liquid separation. In the solid liquid separation process, the solid DCDPS formed by cooling is separated from the carboxylic acid and the water.

Independently of whether the cooling and crystallization is performed continuously or batchwise, the solid-liquid-separation (b) can be carried out either continuously or batchwise, preferably continuously.

If the cooling and crystallization is carried out batchwise and the solid-liquid-separation is carried out continuously at least one buffer container is used into which the suspension withdrawn from the gastight closed vessel is filled. For providing the suspension a continuous stream is withdrawn from the at least one buffer container and fed into a solid-liquid-separation apparatus. The volume of the at least one buffer container preferably is such that each buffer container is not totally emptied between two filling cycles in which the contents of the gastight closed vessel is fed into the buffer container. If more than one buffer container is used, it is possible to fill one buffer container while the contents of another buffer container are withdrawn and fed into the solid-liquid-separation. In this case the at least two buffer containers are connected in parallel.

The parallel connection of buffer containers further allows filling the suspension into a further buffer container after one buffer container is filled. An advantage of using at least two buffer containers is that the buffer containers may have a smaller volume than only one buffer container. This smaller volume allows a more efficient mixing of the suspension to avoid sedimentation of the crystallized DCDPS. To keep the suspension stable and to avoid sedimentation of solid DCDPS in the buffer container, it is possible to provide the buffer container with a device for agitating the suspension, for example a stirrer, and to agitate the suspension in the buffer container. Agitating preferably is operated such that the energy input by stirring is kept on a minimal level, which is high enough to suspend the crystals but prevents them from breakage. For this purpose, the energy input preferably is preferably in the range from 0.2 to 0.5 W/kg, particularly in the range from 0.25 to 0.4 W/kg. Moreover, a stirring device is used which does not show high local energy dissipation input to prevent from attrition of the crystals.

If the cooling and crystallization and the solid-liquid-separation are carried out batchwise the contents of the gastight closed vessel directly can be fed into a solid-liquid-separation apparatus as long as the solid-liquid separation apparatus is large enough to take up the whole contents of the gastight closed vessel. In this case it is possible to omit the buffer container. It is also possible to omit the buffer container when cooling and crystallization and the solid-liquid-separation are carried out continuously. In this case also the suspension directly is fed into the solid-liquid-separation apparatus. If the solid-liquid separation apparatus is too small to take up the whole contents of the gastight closed vessel, also for batchwise operation at least one additional buffer container is necessary to allow to empty the gastight closed vessel and to start a new batch.

If the cooling and crystallization are carried out continuously and the solid-liquid-separation is carried out batchwise the suspension withdrawn from the gastight closed vessel is fed into the buffer container and each batch for the solid-liquid-separation is withdrawn from the buffer container and fed into the solid-liquid-separation apparatus.

The solid-liquid-separation for example comprises a filtration, centrifugation or sedimentation. Preferably, the solid-liquid-separation is a filtration. In the solid-liquid-separation liquid mother liquor comprising carboxylic acid and water is removed from the solid DCDPS and residual moisture containing DCDPS (in the following also termed as “moist DCDPS”) is obtained as product. If the solid-liquid-separation is a filtration, the moist DCDPS is called “filter cake”.

Independently of carried out continuously or batchwise, the solid-liquid-separation preferably is performed at ambient temperature or temperatures below ambient temperature, preferably at ambient temperature. It is possible to feed the suspension into the solid-liquid-separation apparatus with elevated pressure for example by using a pump or by using an inert gas having a higher pressure, for example nitrogen. If the solid-liquid-separation is a filtration and the suspension is fed into the filtration apparatus with elevated pressure the differential pressure necessary for the filtration process is realized by setting ambient pressure to the filtrate side in the filtration apparatus. If the suspension is fed into the filtration apparatus at ambient pressure, a reduced pressure is set to the filtrate side of the filtration apparatus to achieve the necessary differential pressure. Further, it is also possible to set a pressure above ambient pressure on the feed side of the filtration apparatus and a pressure below ambient pressure on the filtrate side or a pressure below ambient pressure on both sides of the filter in the filtration apparatus, wherein also in this case the pressure on the filtrate side must be lower than on the feed side. Further, it is also possible to operate the filtration by only using the static pressure of the liquid layer on the filter for the filtration process. Preferably, the pressure difference between feed side and filtrate side and thus the differential pressure in the filtration apparatus is in the range from 100 to 6000 mbar(abs), more preferred in the range from 300 to 2000 mbar(abs) and particularly in the range from 400 to 1500 mbar(abs), wherein the differential pressure also depends on the filters used in the solid-liquid-separation (b).

To carry out the solid-liquid-separation (b) any solid-liquid-separation apparatus known by the skilled person can be used. Suitable solid-liquid-separation apparatus are for example an agitated pressure nutsche, a rotary pressure filter, a drum filter, a belt filter or a centrifuge. The pore size of the filters used in the solid-liquid-separation apparatus preferably is in the range from 1 to 1000 μm, more preferred in the range from 10 to 500 μm and particularly in the range from 20 to 200 μm.

The apparatus for solid-liquid separation, particularly the filtration apparatus, preferably is made of a nickel-base alloy or stainless steel. Further it is also possible to use coated steel, wherein the coating is made of a material which is resistant against corrosion. If the solid-liquid-separation is a filtration, the filtration apparatus preferably comprises a filter element which is made of a material which has a good or very good chemical resistance. Such materials can be polymeric materials or chemical resistant metals as described above for the used apparatus. Filter elements for example can be filter cartridges, filter membranes, or filter cloth. If the filter element is a filter cloth, preferred materials additionally are flexible, particularly flexible polymeric materials such as those which can be fabricated into wovens. These can for instance be polymers which can be drawn or spun into fibers. Particularly preferred as material for the filter element are polyether ether ketone (PEEK), polyamide (PA) or fluorinated polyalkylenes, for example ethylene chlorotrifluoroethylene (ECTFE), polytetrafluoroethylene (PTFE), polyvinylidene fluoride (PVDF), fluorinated ethylene-propylene (FEP).

Particularly preferably, cooling and crystallization is carried out batchwise and the solid-liquid-separation is operated continuously.

If the solid-liquid-separation is a filtration, it is possible to carry out the following washing of the filter cake in the filtration apparatus, independently of whether the filtration is operated continuously or batchwise. After washing, the filter cake is removed as product.

In a continuous solid-liquid-separation process, the moist DCDPS can be removed continuously from the solid-liquid-separation apparatus and afterwards the washing of the moist DCDPS takes place. In the case the solid-liquid separation is a filtration and a continuous belt filter is used, it is preferred to filtrate the suspension, to transport the thus originating filter cake on the filter belt and to wash the filter cake at a different position in the same filtration apparatus. However, if the solid-liquid separation is a continuously operated filtration, it is preferred to carry out the solid-liquid-separation and the subsequent washing in the same apparatus.

If the solid-liquid separation is a filtration process, it is further also possible to operate the filtration semi-continuously. In this case the suspension is fed continuously into the filtration apparatus and the filtration is performed for a specified process time. Afterwards the filter cake produced during the filtration is washed in the same filtration apparatus. The process time for performing the filtration for example may depend on the differential pressure. Due to the increasing filter cake the differential pressure in the filtration apparatus increases. To determine the process time for the filtration, it is for example possible to define a target differential pressure up to which the filtration is carried out in a first filtration apparatus. Thereafter the suspension is fed into a second or further filtration apparatus in which filtration is continued. This allows to continuously perform the filtration. In those apparatus where the filtration is completed, the filter cake can be washed and withdrawn after finishing the washing. If necessary, the filtration apparatus can be cleaned after the filter cake is withdrawn. After the filter cake is withdrawn and the filter apparatus is cleaned when necessary, the filtration apparatus can be used again for filtration. If the washing of the filter cake and the optional cleaning of the filtration apparatus needs more time than the time for the filtration in one filtration apparatus at least two filtration apparatus are used to allow continuous feeding of the suspension in a filtration apparatus while in the other apparatus the filter cake is washed or the filtration apparatus are cleaned.

In each filtration apparatus of the semi-continuous process, the filtration is carried out batchwise. Therefore, if the filtration and washing are carried out batchwise, the process corresponds to the process in one apparatus of the above described semi-continuous process.

After the solid-liquid separation is completed, the moist DCDPS is washed to remove remainders of the carboxylic acid and further impurities, for example undesired by-products which formed during the process for producing the DCDPS.

Washing thereby is carried out in at least two phases. In a first phase, the moist DCDPS is washed with an aqueous base which is followed by washing with water in a second phase.

To remove the remainders of the carboxylic acid from the moist DCDPS, the aqueous base used for washing in the first phase preferably is an aqueous alkali metal hydroxide, for example aqueous potassium hydroxide or sodium hydroxide, particularly sodium hydroxide. If an alkali metal hydroxide is used as aqueous base, the aqueous alkali metal hydroxide preferably comprises from 1 to 50 wt % alkali metal hydroxide based on the total amount of aqueous alkali metal hydroxide, more preferred from 1 to 20 wt % alkali metal hydroxide based on the total amount of aqueous alkali metal hydroxide and particularly from 2 to 10 wt % alkali metal hydroxide based on the total amount of aqueous alkali metal hydroxide. This amount is sufficient for properly washing the moist DCDPS.

By using the aqueous alkali metal hydroxide, the anion of the carboxylic acid reacts with the alkali metal cation of the alkali metal hydroxide forming an organic salt and water. In difference to the carboxylic acid which generally is not soluble in water and depending on the carboxylic acid also even may be immiscible with water, the organic salt formed by reaction with the aqueous base is soluble in water and thus remainders which are not removed with the aqueous alkali metal hydroxide and the water formed by the reaction can be removed from the moist DCDPS by washing with water. This allows to achieve DCDPS as product which contains less than 1 wt %, preferably less than 0.7 wt % and particularly less than 0.5 wt % organic impurities.

For obtaining DCDPS with such a small content of organic impurities, the amount of the aqueous base, particularly the alkali metal hydroxide used for the washing in the first phase preferably is in a range from 0.5 to 10 kg per kg dry DCDPS, more preferred in a range from 1 to 6 kg per kg dry DCDPS and particularly in a range from 2 to 5 kg per kg dry DCDPS.

As the water of the aqueous base and the water produced by the reaction of the anion of the base with the carboxylic acid generally is not sufficient to remove all of the organic salt and as further part of the aqueous base may stay in the moist DCDPS, the moist DCDPS is washed with water in the second phase. By washing with water, remainders of the organic salt and of the aqueous base which did not react are removed. The water then can be easily removed from the DCDPS by usual drying processes known to a skilled person to obtain dry DCDPS as product. Alternatively, it is possible to use the water wet DCDPS which is obtained after washing with water in subsequent process steps.

The amount of water used for washing in the second phase preferably is chosen such that the aqueous base remaining in the DCDPS after washing with the aqueous base is removed. This can be achieved for example by measuring the pH value of the moist DCDPS. Washing is continued until the DCDPS is neutral which means a pH value in the range from 6.5 to 7.5, preferably in the range from 6.8 to 7.2 and particularly in the range from 6.9 to 7.1. This can be achieved by using water for washing after washing with the aqueous base in an amount which preferably is in the range from 0.5 to 10 kg per kg dry DCDPS, more preferred in the range from 1 to 7 kg per kg dry DCDPS and particularly in the range from 1 to 5 kg per kg dry DCDPS. Using such an amount of water for washing in the second phase has the advantage that the amount of waste water which has to be withdrawn from the process and passed into a purification plant for cleaning can be kept on a very low level.

The washing with water in the second phase preferably is carried out in two washing steps. In this case, it is particularly preferred to use fresh water for the washing in the second washing step and to use the water which has been used in the second washing step in the first washing step. This allows the amount of water which is used for washing in total to be kept low.

If the solid-liquid-separation is a filtration, it is possible to carry out the following washing of the filter cake in the filtration apparatus, independently of whether the filtration is operated continuously or batchwise. After washing, the filter cake is removed as product.

Besides carrying out filtration and washing of the filter cake in one apparatus, it is also possible to withdraw the filter cake from the filtration apparatus and wash it in a subsequent washing apparatus. If the filtration is carried out in a belt filter, it is possible to convey the filter cake on the filter belt into the washing apparatus. For this purpose, the filter belt is designed in such a way that it leaves the filtration apparatus and enters into the washing apparatus. Besides transporting the filter cake on a filter belt from the filtration apparatus into the washing apparatus it is also possible to collect the filter cake with a suitable conveyor and feed the filter cake from the conveyor into the washing apparatus. If the filter cake is withdrawn from the filtration apparatus with a suitable conveyor the filter cake can be withdrawn from the filtration apparatus as a whole, or in smaller pieces such as chunks or pulverulent. Chunks for instance arise if the filter cake breaks when it is withdrawn from the filtration apparatus. To achieve a pulverulent form, the filter cake usually must be comminuted. Independently from the state of the filter cake, for washing the filter cake is brought into contact with the aqueous base and subsequently with water. For example, the filter cake can be put on a suitable tray in the washing apparatus and the aqueous base flows through the tray and the filter cake. Further it is also possible to break the filter cake into smaller chunks or particles and to mix the chunks or particles with the aqueous base. Subsequent the thus produced mixture of chunks or particles of the filter cake and the aqueous base is filtrated to remove the aqueous base. If the washing is carried out in a separate washing apparatus, the washing apparatus can be any suitable apparatus. Preferably the washing apparatus is a filter apparatus which allows to use a smaller amount of aqueous base and to separate the aqueous base from the solid DCDPS in only one apparatus. However, it is also possible to use for example a stirred tank as washing apparatus. In this case it is necessary to separate the aqueous base from the washed DCDPS in a following step, for example by filtration or centrifugation. After the washing with the aqueous base, the washing with water is carried out in the same way. Thereby, for washing with the aqueous base and the washing with water only one apparatus can be used or the washing with the aqueous base and the subsequent washing with water are carried out in different apparatus.

If the solid-liquid-separation (b) is carried out by centrifugation, depending on the centrifuge it might be necessary to use a separate washing apparatus for washing the moist DCDPS. However, usually a centrifuge can be used which comprises a separation zone and a washing zone or the washing can be carried out after centrifuging in the centrifuge.

Washing of the moist DCDPS preferably is operated at ambient temperature. It is also possible to wash the moist DCDPS at temperatures different to ambient temperature, for instance above ambient temperature. If the washing is carried out in the filtration apparatus, for washing the filter cake a differential pressure must be established. This is possible for example by feeding the aqueous base in the first phase and the water in the second phase for washing the filter cake at a pressure above ambient pressure and withdraw the aqueous base and the water, respectively, after passing the filter cake at a pressure below the pressure at which the aqueous base and the water are fed, for example at ambient pressure. Further it is also possible to feed the aqueous base and the water for washing the filter cake at ambient pressure and withdraw the aqueous base and the water after passing the filter cake at a pressure below ambient pressure.

Particularly the aqueous base which was used for washing the moist DCDPS contains either carboxylic acid or the organic salt of the carboxylic acid. To reduce the amount of carboxylic acid which is withdrawn with the water and subjected to purification in a purification plant and thereby completely removed, according to the invention, in one alternative the aqueous base after being used for washing is mixed with a strong acid.

In a second alternative, the aqueous base after being used for washing is mixed with at least a part of the carboxylic acid comprising filtrate obtained in (b) and a strong acid. In this case it is possible, to firstly mix the aqueous base after being used for washing and at least a part of the carboxylic acid comprising filtrate and then mix this mixture with the strong acid or to mix the aqueous base after being used for washing, at least a part of the carboxylic acid comprising filtrate and the strong acid simultaneously.

By mixing with the strong acid, the organic salt which formed during washing with the aqueous base reacts with the strong acid forming the carboxylic acid from the anion of the organic salt and a second salt from the anion of the strong acid. The strong acid preferably is selected such that the second salt which forms has a good solubility in water and a poor solubility in the carboxylic acid. In this context “good solubility” means at least 20 g per 100 g solvent can be dissolved and “poor solubility” means that less than 5 g per 100 g solvent can be dissolved in the solvent.

The poor solubility of the second salt in the carboxylic acid has the effect that the carboxylic acid which can be recovered comprises less than 3 ppm wt % impurities based on the total mass of the carboxylic acid. This allows further use of the carboxylic acid without further purification steps.

Depending on the aqueous base which is used for the washing of the moist DCDPS, the strong acid preferably is sulfuric acid or a sulfonic acid, like paratoluene sulfonic acid or alkane sulfonic acid, for example methane sulfonic acid. If the aqueous base is an alkali metal hydroxide, the strong acid particularly preferably is sulfuric acid.

Mixing of the aqueous base after being used for washing and the strong acid or mixing of the aqueous base, at least a part of the carboxylic acid comprising filtrate and the strong acid can be carried out in any mixer known to a skilled person. Suitable mixers for mixing the aqueous base after being used for washing and the strong acid for example is a static mixer, a tube, a dynamic mixer like a mixing pump, or a stirred vessel. If in a first step the aqueous base and at least a part of the carboxylic acid comprising filtrate are mixed and then the strong aced is mixed to this mixture, a first mixer can be used for mixing the aqueous base and the carboxylic acid comprising filtrate and a second mixer for mixing this mixture with the strong acid. Particularly if a stirred vessel is used, it is possible to firstly add the aqueous base and the carboxylic acid comprising filtrate, start mixing and then to add the strong acid. If the aqueous base, at least a part of the carboxylic acid and the strong acid are mixed simultaneously, all three components are added to the same mixer at the same time. If a stirred vessel is used for mixing, it is also possible to feed the components into the stirred vessel and to start mixing after all components are fed into the stirred vessel.

To allow reusing the carboxylic acid, the carboxylic acid has to be separated from the aqueous phase. This is carried out in the phase separation (e). The carboxylic acid separated by the phase separation (e) can be used in any process in which a respective carboxylic acid is used. However, it is particularly preferred to recycle the carboxylic acid into the process for producing the DCDPS. If the carboxylic acid contains impurities after being separated off in (e), it is further possible, to subject the carboxylic acid to additional purifying steps like washing or distillation to remove high boiling or low boiling impurities.

Due to the comparatively small amount of carboxylic acid in the aqueous base after being mixed with the strong acid, if not at least a part of the carboxylic acid is mixed with the aqueous base or only a part of the carboxylic acid comprising filtrate, it is possible to add at least a part of the carboxylic acid comprising filtrate to the aqueous base mixed with the strong acid before carrying out the phase separation. This allows to improve the efficiency of the phase separation.

Particularly in case the cooling and crystallization is carried out in the gastight closed vessel by adding water and reducing the pressure, the carboxylic acid comprising filtrate additionally contains water. To allow reuse of the carboxylic acid in this case also the filtrate must be subject to a phase separation. Mixing the aqueous base mixed with the strong acid and the carboxylic acid comprising filtrate or mixing the aqueous base, the carboxylic acid comprising filtrate and the strong acid in this case has the additional advantage that only one phase separation has to be carried out for separating the organic carboxylic acid from the aqueous phase.

Depending on the amounts of organic phase and aqueous phase and the process used for phase separation, it may be necessary to increase the amount of the aqueous phase in the mixture. This can be achieved for example by circulating at least a part of the aqueous phase through the phase separation apparatus and the mixing device. Preferably, the phase separation apparatus and the mixing device are combined in one apparatus, particularly a mixer-settler and the at least part of the aqueous phase is circulated through the mixer-settler. For circulating at least a part of the aqueous phase through the phase separation apparatus and the mixing device, preferably the mixer-settler, the at least part of the aqueous phase is branched off the total aqueous phase withdrawn from the phase separation apparatus and mixed with the carboxylic acid comprising filtrate and the aqueous base mixed with the strong acid before this mixture is subjected to the phase separation again.

Mixing of the carboxylic acid comprising filtrate, the aqueous base mixed and the strong acid and—if applicable—with the part of the aqueous phase to be circulated can be carried in a separate mixing device or preferably in the mixing part of a mixer-settler in which also the phase separation takes place. Mixing and phase separation can be carried out batchwise or continuously. If mixing and phase separation are carried out continuously and the mixture flows through the mixer settler, for mixing the several streams, preferably a coalescing aid is placed in the mixing part of the mixer-settler. Such a coalescing aid for example is a packed layer like a structured packing or a random packing. Further, a knitted mesh or a coalescer can be used as coalescing aid. Filling bodies used for the random packing can be for example Pall®-rings, Raschig®-rings or saddles.

To avoid particle clogging, after filtration the mother liquor can be used for flushing the outlet for the aqueous base of the filter.

If the phase separation is carried out batchwise, it is possible to feed all streams separately into a mixer-settler, mix them, for example by agitating like stirring, then stop stirring and let the phases separate. After phase separation is completed, the aqueous phase and the organic phase can be withdrawn from the mixer-settler separately.

Further, independently of carrying out the phase separation batchwise or continuously, it is also possible to mix the streams before feeding into a phase separation apparatus. Mixing in this case can be carried out in a static or dynamic mixer to which the streams are added or preferably by feeding all streams into one tube and mixing results from turbulence in the stream. If a static mixer is used, the mixer may contain a coalescing aid as described above.

Besides feeding the part of the aqueous phase which was branched off for circulating into the phase separation apparatus before or after mixing with the carboxylic acid comprising filtrate and the aqueous base after being mixed with the strong acid, it is also possible to recirculate the part of the aqueous phase into the mixing of the aqueous base with the strong acid.

Additionally or alternatively, it is also possible to increase the amount of the aqueous phase by feeding at least a part of the water which was used for washing in the second phase after the washing with the aqueous base, into the phase separation. By feeding at least a part of this water into the phase separation even traces of organic impurities, particularly carboxylic acid which may still be comprised in the DCDPS after washing with the aqueous base can be regained.

To reduce the amount of water which is disposed, it is further possible and preferred, to use at least a part of the water which was used for washing the moist DCDPS in the second phase for producing the aqueous base which is used for washing the moist DCDPS in the first phase.

The DCDPS obtained by this purifying process for example can be used as starting material for producing sulfone polymers, particularly for producing polyarylene(ether)sulfone.

Each process step described above can be carried out in only one apparatus or in more than one apparatus depending on the apparatus size and the amount of compounds to be added. If more than one apparatus is used for a process step, the apparatus can be operated simultaneously or—particularly in a batchwise operated process—at different time. This allows for example to carry out a process step in one apparatus while at the same time another apparatus for the same process step is maintained, for example cleaned. Further, in that process steps where the contents of the apparatus remain for a certain time after all components are added, for example the oxidization reaction or the cooling steps, it is possible after feeding all compounds in one apparatus to feed the components into a further apparatus while the process in the first apparatus still continues. However, it is also possible to add the components into all apparatus simultaneously and to carry out the process steps in the apparatus also simultaneously.

An illustrative embodiment of the invention is shown in the FIGURE and explained in more detail in the following description.

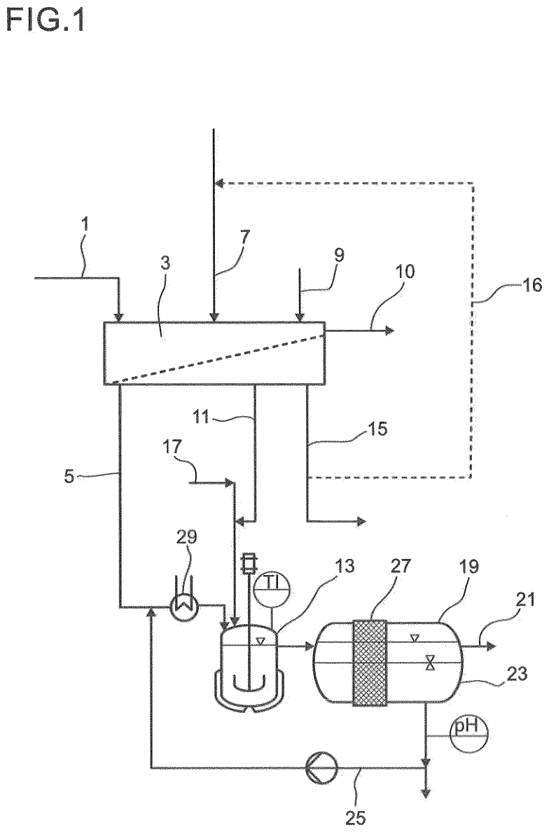

FIG. 1 shows a flow diagram of an embodiment of the inventive process.

In FIG. 1 the process for purifying a DCDPS comprising suspension is shown in a flow diagram.

A suspension 1 comprising solid DCDPS in a carboxylic acid and optionally water is fed into a solid-liquid separation apparatus 3, for example a filtration apparatus. The filtration apparatus can be an agitated pressure nutsche, a rotary pressure filter, a drum filter or a belt filter. Besides a filtration apparatus, the solid-liquid separation apparatus also can be a centrifuge.

In the solid-liquid separation apparatus 3 the suspension is separated into moist DCDPS and a carboxylic acid and optionally water comprising filtrate 5 which is withdrawn from the solid-liquid separation apparatus.

After completion of the solid-liquid separation, the moist DCDPS is washed in two phases. In a first phase, the moist DCDPS is washed with an aqueous base 7 and after completing washing with the aqueous base, in a second phase the moist DCDPS is washed with water 9. The aqueous base for washing in the first phase preferably is aqueous alkali metal hydroxide, particularly sodium hydroxide. The moist DCDPS after washing with aqueous base and water is withdrawn from the solid-liquid separation apparatus 3 as product stream 10.

The solid-liquid separation and the two washing phases can be carried out in only one apparatus or in different apparatus for solid-liquid separation and washing. If a continuous belt filter is used for solid-liquid separation and washing, the moist DCDPS is transported on the belt from the solid-liquid separation to the position where the washing takes place. If a solid liquid apparatus is used in which the moist DCDPS cannot be transported on the filter, solid-liquid separation and washing can be carried out in the same apparatus in succession. In this case, the moist DCDPS forming a filter cake is removed from the filter after completion of the solid-liquid separation and the washing phases.

After being used for washing, the aqueous base 11 is fed into a vessel 13. The water after use is withdrawn from the process by drainage line 15. Further it is possible to use at least a part of the water after use for diluting the aqueous base 7. This is exemplary shown with dashed line 16.

By washing the moist DCDPS with the aqueous base, remainders of the carboxylic acid react with the base forming a carboxylate. To reduce the amount of waste and to increase the amount of carboxylic acid which can be reduced, after being used for washing, the aqueous base is mixed with a strong acid 17. The strong acid reacts with the carboxylate forming a salt and the carboxylic acid. The mixing of the aqueous base after being used and the strong acid can take place in a stirred tank, a tube or a static mixer. In the embodiment according to the FIGURE, the strong acid is added to the aqueous base in the line through which the aqueous base is fed into the vessel 13. The vessel 13 is a stirred tank in which the components fed into the vessel 13 are agitated, particularly stirred. Therefore, the reaction of the strong acid with the carboxylate in the aqueous base takes place in the vessel 13.

According to the embodiment shown in the FIGURE, also the filtrate 5 is fed into the vessel 13 and mixed with the strong acid and the aqueous base.

As an alternative, it is also possible to add the aqueous base 11, the strong acid 17 and the filtrate 5 via separate feed lines into the vessel 13. This allows for mixing the aqueous base 11 and the filtrate 5 in a first step and to add the strong acid 17 to this mixture.

Besides the embodiment shown in the FIGURE, it is also possible to complete the reaction of the strong acid and the carboxylate in the aqueous base before feeding into a buffer container into which also the filtrate is fed. Preferably, the buffer container is equipped with a mixing device for mixing the aqueous base and the filtrate.

To improve the phase separation, the filtrate 5 can be heated in a heat exchanger 29. Preferably, the filtrate 5 is heated to a temperature in the range from 30 to 50° C. A further advantage of heating the filtrate 5 to such a temperature is that precipitation of solids can be avoided.

From the vessel 13 or alternatively the buffer container, the mixture of the filtrate and the aqueous base is fed into a phase separation apparatus 19. In the phase separation apparatus 19, the mixture is separated into an organic phase 21 comprising the carboxylic acid and an aqueous phase 23 in which the salt formed from the anion of the strong base and the cation of the aqueous base is solved. The organic phase 21 is withdrawn from the phase separation apparatus 19 and the carboxylic acid can be reused. If necessary, it is possible to subject the organic phase to further purification steps before reusing the carboxylic acid.

Due to the small amount of aqueous phase compared to the amount of organic phase and to facilitate the phase separation, a part of the aqueous phase is recycled into the vessel 13 via recirculation line 25. Besides recycling into the vessel 13, alternatively it is also possible to recycle the aqueous phase directly into the phase separation apparatus 19. The part of the aqueous phase 23 which is not recycled is withdrawn from the process and disposed, optionally after being purified.

To facilitate the phase separation in the phase separation in a continuously operated phase separation apparatus 19 as shown in the FIGURE, a coalescing aid 27 is provided. The coalescing aid for example is a random packing, for example a layer of Pall® rings, Raschig® rings or saddles or a structured packing.

### EXAMPLES

### Example 1

4902 g suspension comprising 1547 g crystallized DCDPS, 811 g water and 2544 g n-heptanoic acid were filled on a laboratory nutsche. A pressure of 500 mbar(abs) was set to the filtrate side of the nutsche for 60 seconds to carry out the filtration. After finishing the filtration the thus obtained filter cake was dried 30 seconds with dry air.

Afterwards the filter cake was washed with 2 kg of diluted NaOH 5%. For washing a pressure of 750 mbar(abs) were set to the filtrate side of the nutsche.

Washing with diluted NaOH was followed by washing with 1.5 kg water. For washing with water a pressure of 500 mbar(abs) were set to the filtrate side of the nutsche. Subsequently the filter cake was dried for 30 seconds drying with air.

After washing and drying, the content of carboxylic acid in the filter cake was 0.24 wt %. The final filter cake mass was 1369 g.

The mother liquor obtained in the filtration process was subjected to a phase separation. By phase separation, 482 g aqueous phase and 2712 g organic phase were obtained.

### Example 2

1000 g DCDPSO having an APHA-number of 100 were dissolved in 3000 g n-heptanoic acid. This solution was heated to 90° C. Then 1.3 g sulfuric acid and 197 g H₂O₂ were added over a period of 3 h and 40 min for oxidizing the DCDPSO to obtain DCDPS. To the solution obtained by the oxidation reaction, 794 g water were added.

After adding the water, the thus obtained solution was cooled to crystallize the DCDPS obtained in the oxidation reaction. For cooling, the pressure was continuously reduced over a period of 5 h. Due to the pressure reduction, the water started to evaporate. The evaporated water was condensed and returned into the solution. By this process the temperature was reduced by 83 K. By this cooling process the DCDPS crystallized and a suspension formed. This suspension was separated into a DCDPS containing filter cake and a mother liquor by solid-liquid separation.

The filter cake was washed with 1.3 kg diluted NaOH 5%. After washing with the diluted NaOH, the filter cake was washed two times with 1.3 kg water each. After washing the DCDPS was dried at 60° C. for 16 h. The thus obtained DCDPS had an APHA-number of 30 and contained 0.16 wt % n-heptanoic acid.

The pressures and the operation times at which the solid-liquid separation and washing steps were carried out were the same as described for example 1.

After being used for washing, the diluted NaOH was mixed with the mother liquor obtained by the solid-liquid separation. 175.2 g 50% sulfuric acid were added to the mixture of diluted NaOH and mother liquor. The thus obtained liquid mixture was subjected to a phase separation to obtain an aqueous phase and an organic phase. The aqueous phase and the waste water of the water washing steps were mixed. This resulted in 4.8 L cumulated waste water which had a TOC of 2700 mg/L which corresponds to 13 g organic compounds which were primarily the carboxylic acid. This shows that only 0.43% of the carboxylic acid which was used for dissolving the DCDPSO were withdrawn from the process by the waste water.

The organic phase which essentially comprised heptanoic acid was worked-up for purifying the heptanoic acid and the heptanoic acid was recycled into the production process of DCDPS.

### LIST OF REFERENCE NUMERALS

1 suspension
3 solid-liquid separation apparatus
5 filtrate
7 aqueous base
9 water
10 product stream
11 aqueous base after being used for washing
13 vessel
15 drainage line
17 strong acid
19 phase separation apparatus
21 organic phase
23 aqueous phase
25 recirculation line
27 coalescing aid

1.-14. (canceled)

15. A process for purifying 4,4′-dichlorodiphenyl sulfone comprising:
(a) providing a suspension comprising particulate 4,4′-dichlorodiphenyl sulfone in carboxylic acid,
(b) carrying out a solid-liquid separation of the suspension to obtain residual moisture containing 4,4′-dichlorodiphenyl sulfone and a carboxylic acid comprising filtrate,
(c) washing the residual moisture containing 4,4′-dichlorodiphenyl sulfone with an aqueous base and then with water,
(d) mixing the aqueous base after being used for washing with a strong acid and then mixing at least a part of the carboxylic acid comprising filtrate with the aqueous base mixed with the strong acid, or mixing the aqueous base after being used for washing, at least a part of the carboxylic acid comprising filtrate and a strong acid,
(e) carrying out a phase separation in which an aqueous phase and an organic phase comprising the carboxylic acid are obtained.

16. The process according to claim 15, wherein the carboxylic acid is a linear C₆ to C₁₀ carboxylic acid.

17. The process according to claim 15, wherein the carboxylic acid is selected from the group consisting of n-hexanoic acid, n-heptanoic acid.

18. The process according to claim 15, wherein the aqueous base is an aqueous alkali metal hydroxide.

19. The process according to claim 18, wherein the aqueous alkali metal hydroxide comprises from 1 to 50 wt % alkali metal hydroxide based on the total amount of aqueous alkali metal hydroxide.

20. The process according to claim 15, wherein the amount of the aqueous base used for washing is in the range from 0.5 to 10 kg per kg dry 4,4′-dichlorodiphenyl sulfone.

21. The process according to claim 15, wherein the amount of water used for washing after washing with the aqueous base is in the range from 0.5 to 10 kg per kg dry 4,4′-dichlorodiphenyl sulfone.

22. The process according to claim 15, wherein the solid-liquid separation (b) and the washing (c) are carried out in one apparatus.

23. The process according to claim 15, wherein the strong acid is sulfuric acid or alkane sulfonic acid.

24. The process according to claim 15, wherein the carboxylic acid comprising filtrate and the aqueous base after being used for washing are mixed before mixing with the strong acid.

25. The process according to claim 15, wherein at least a part of the aqueous phase obtained in the phase separation (e) is recirculated into the mixing (d) of the aqueous base with the strong acid.

26. The process according to claim 15, wherein at least a part of the water after being used for washing is used for producing the aqueous base.

27. The process according to claim 15, wherein at least a part of the carboxylic acid obtained in (e) is used in a process for producing 4,4′-dichlorodiphenyl sulfone by oxidation of 4,4′-dichlorodiphenyl sulfoxide in the presence of a carboxylic acid

28. The process according to claim 15, wherein the 4,4′-dichlorodiphenyl sulfone is used as starting material for producing sulfone polymers, particularly polyarylene(ether)sulfone.
